(19) United States
(12) Patent Application Publication
(10) Pub. No.: US 2003/0199739 A1
(43) Pub. Date: Oct. 23, 2003

(54) Printing device for personal medical monitors

(76) Inventors: Tim H. Gordon, Rivervale, NJ (US); Scott W. Gisler, Pine Brook, NJ (US); Michael Puma, Midland Park, NJ (US); Raymond L. Yao, Hoboken, NJ (US); Pierre Cassigneul, Flemington, NJ (US)

Correspondence Address: BECTON, DICKINSON AND COMPANY, 1 BECTON DRIVE, FRANKLIN LAKES, NJ, 07417-1880, US

(21) Appl. No.: 10/316,679

(22) Filed: Dec. 11, 2002

Related U.S. Application Data
(60) Provisional application No. 60/339,809, filed on Dec. 17, 2001.

(51) Int. Cl. A61B 5/00

(52) U.S. Cl. 600/300

(57) ABSTRACT

A printing device is provided to increase the benefit to patients and healthcare professionals of personal medical monitoring devices, such as blood glucose meters. The printing device receives and analyzes data from a personal medical monitoring device, such as a blood glucose meter, and generates an output based on analysis of the data. The output may include a log of the data entries, or graphical representations of the data, such as pie charts, line charts, histograms, and the like. Furthermore, the data analysis performed by the device can comprise applying therapy rules to the data. In this manner, the device is capable of and providing therapy recommendations in the output based on the analysis of data and application of therapy rules, and to thereby provide therapy guidance for a patient and their respective health care professional.

### FIELD OF THE INVENTION

The present invention relates to personal medical monitors, such as blood glucose meters (BGMs). In particular, the present invention relates to a printing device for retrieving and analyzing data from a personal medical monitor, such as a body fluid chemistry analyzer, and for formatting the data and printing a result based on the data. The output can include a log of data entries from the personal medical monitor, and can also include graphical representations derived from the data, such as pie charts, trends, histograms, and the like. Furthermore, a printing device according to the present invention can apply therapy rules, and include therapy recommendations in a printout based on analysis of existing data, in order to provide therapy guidance to both a healthcare professional and a patient.

### BACKGROUND OF THE INVENTION

The value of a medical device that can generate and store clinical data is realized in the subsequent utilization of that data to affect clinical outcomes. One example is the utilization of self monitored blood glucose (SMBG) data for diabetes management. Numerous factors presently constrain more effective use of SMBG data generated by blood glucose meters. Both patients and health care providers are constrained by the limitations of current hardware, data analysis and presentation, time, training, and application of therapy principles, to the valuable data generated by patients every day.

Current SMBG data utilization is accomplished with the use of a meter's display, review of a patient's hand generated logbook, or by connecting the meter and downloading its data into a PC based software program. Unfortunately, each of these methods has inherent shortfalls. Data utilization based on the meter display and patient logbook is better than not using the data at all, but is limited by the clinical knowledge and analytical capabilities of the user. Additionally, a hand generated logbook is prone to frequent errors, thus the data lacks integrity.

Data utilization based on current PC download systems enhance the user's ability to analyze the data to some extent. However, current PC systems fall short in at least two respects. First, current systems present ease of use issues. The user must be familiar with connecting and configuring the system, as well as accessing the system and presenting data in a meaningful and useful format. Second, current systems provide only limited direction on how to apply the analyzed data to enhance clinical decisions.

### SUMMARY OF THE INVENTION

The present invention overcomes the above disadvantages and other advantages are realized by providing a system according to the present invention. In one embodiment of the present invention, an output device, such as a printer, is provided with an interface for receiving a body fluid chemistry analyzer, such as a blood glucose monitor. Data is transferred from the blood glucose monitor to the output device through the interface. The data is analyzed and formatted into graphical representations, which are printed for evaluation by both the patient and a health care provider. In another embodiment, therapy rules are applied to the data by the output device, and therapy recommendations are included in the printout by the output device based on the data to provide therapy guidance to both a health care provider and a patient.

### BRIEF DESCRIPTION OF THE DRAWINGS

The invention will be further understood with reference to the attached drawing figures, in which:

FIG. 1 illustrates a first embodiment of an exemplary system according to the present invention;

FIG. 2 illustrates a second embodiment of an exemplary system according to the present invention;

FIG. 3 illustrates a third embodiment of an exemplary system according to the present invention;

FIG. 4 is a block diagram of an exemplary embodiment of a system according to the present invention;

FIG. 5 is a flowchart illustrating an exemplary method according to an embodiment of the present invention;

FIGS. 6A-6C illustrate alternate embodiments of a printing device according to the invention; and

FIG. 7 is a flow chart illustrating the functionality of a processor adapted to control a printing device according to an embodiment of the present invention.

Throughout the drawing figures, it will be understood that like numerals refer to like structures or steps.

### DETAILED DESCRIPTION OF THE PREFERRED EMBODIMENTS

The following description of the presently contemplated best mode of practicing the invention and preferred embodiments thereof is not to be taken in a limiting sense, but is provided merely for the purpose of describing the general principles of the invention.

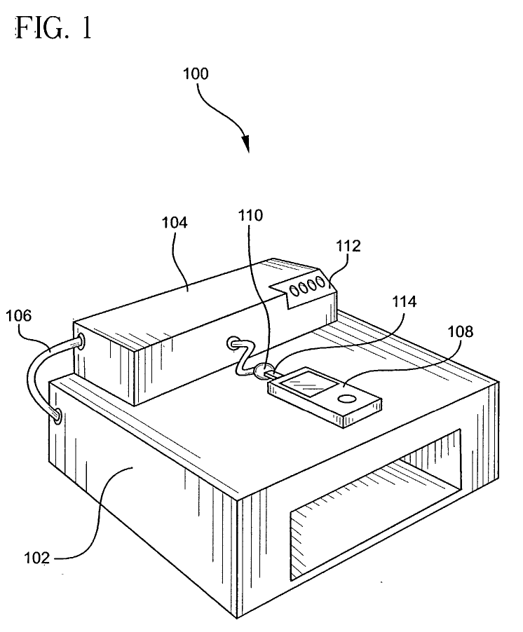

FIG. 1 illustrates a first embodiment of a system 100 according to the present invention. The system comprises a printer 102 connected to an electronics package 104 by a printer connector cable 106. The system further comprises a personal medical monitor 108, such as a blood glucose meter, connected to the electronics package 104 by an interface cable 110. The electronics package 104 has status indicators 112, which are LEDs for instance, which are adapted to provide visual feedback on the status of the system. The status indicators 112 indicate, among other things, whether the system 100 is in an idle ready state, whether the system 100 is connected to a personal medical monitor 108, whether the system 100 is presently printing, or whether the printer 102 needs maintenance. The personal medical monitor 108 has an interface connector 114 which is adapted to be connected to the interface cable 110 in order to facilitate data transfer between the personal medical monitor and the electronics package 104.

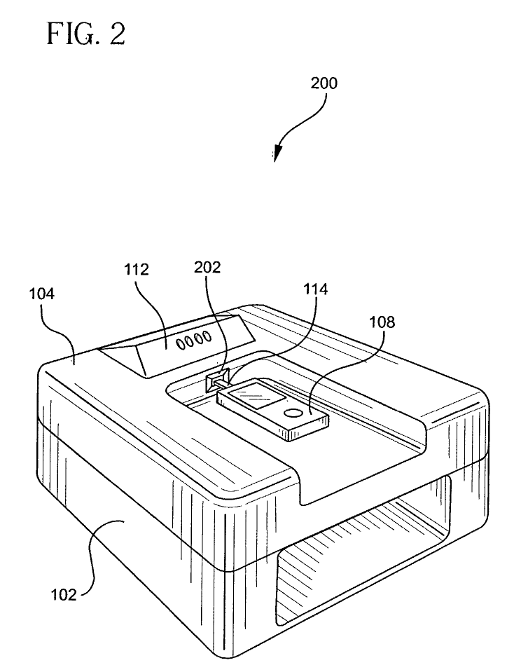

FIG. 2 illustrates a second embodiment of a system 200 according to the present invention. In this embodiment, the electronics package 104 is integrated into the body of the printer 102. Furthermore, the interface cable 110 is replaced by an interface 202 which is also integrated into the body of the printer 102. The interface or port 202 (which can consist of a plug-and-socket arrangement, for example) facilitates data transfer from the personal medical monitor 108 through the interface connector 114 of the personal medical monitor 108 when the personal medical monitor 108 is connected to the interface 202.

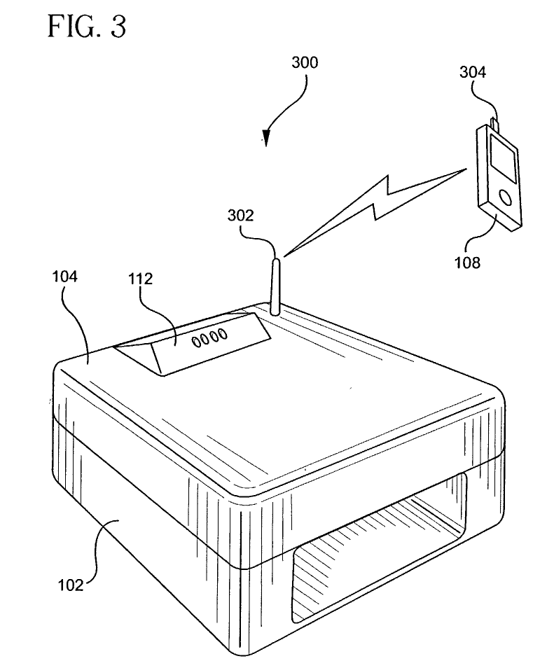

FIG. 3 illustrates a third embodiment of a system 300 according to the present invention. In this embodiment, the printer 102 and personal medical monitor 108 are provided with antennas 302, 204 to facilitate wireless data transfer between the personal medical monitor 108 and the electronics package 104 of the printer 102.

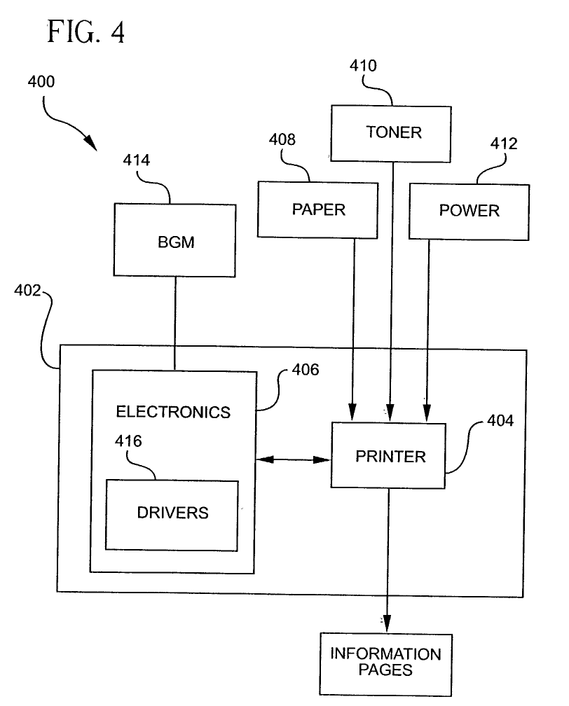

FIG. 4 is a block diagram of a system 400 according to the present invention. The output device 402 preferably comprises a printer 404 and electronics 406. The printer 404 preferably receives paper 408, toner or ink 410 and power 412. The paper 480 can preferably be plain paper in a standard format, such as 8.5″×11″, or A4 sized paper. The paper also may be printed letterhead paper, or have other pre-printed information on it, such as promotional or instructional information, which is not dependent on the data. Of course, one of skill in the art will readily appreciate that the invention is not limited to any particular form factor of paper, and furthermore, the invention is contemplated to be useful in connection with a wide variety of printing and display technologies. The printer 404 is preferably a laser printer, and uses toner 410 to print pages. However, the invention is not limited to laser printers, and any printer such as an inkjet printer, or any other type of output device, including CRT and LCD displays, is considered to be within the scope of the invention. The printer 404 preferably uses standard 120V AC power 412, such as is available in most wall outlets. However, other power sources may be used, as would be convenient and appropriate in a given situation, and any appropriate power supply is considered to be within the scope of the invention. Furthermore, the output device 402 could be powered by batteries (not shown) in lieu of an outside power source, or as a back-up power source.

The electronics 406 perform a number of tasks. Among them, the electronics 406 manage the transfer of data from a blood glucose monitor 414 when a blood glucose monitor 414 is connected to the device 402. Preferably, the electronics are adapted to recognize when a blood glucose monitor 414 has been connected to the output device 402, and begin effectuating a data transfer without any intervention by the user. In the preferred embodiment, the output device 402 has an interface 202 incorporated therein, comprising a physical interface for receiving data from the blood glucose monitor 414. The blood glucose monitor 414 has a corresponding interface 114 adapted to fit the interface 202 of the output device 402, and the user simply inserts the blood glucose monitor 414 into the interface of the output device 402 to begin a data transfer and print operation.

In a preferred embodiment of the invention, the printer 404 has any necessary driver software and configuration software required to interface with a bodily fluid chemistry analyzer pre-installed prior to delivery to an intended user, such as a health care professional's office. This is referred to herein as being pre-configured. Thus, in this embodiment, once the printer 404 is delivered to the user's location, all that is required to use the device is to power it up and connect a blood fluid chemistry analyzer. The driver and configuration software 416 are preferably contained in the electronics 406 of the printer as shown in FIG. 4. Alternatively, they could be stored on a computer readable media, which is adapted to be accessed by the electronics 406.

In another embodiment, the output device 402 comprises a wireless transceiver (not shown) for communicating with a blood glucose monitor 414 having wireless communication ability. The output device 402 is outfitted with a Bluetooth or WiFi (IEEE 802.11b protocol) wireless device, or the like, which provides an alert to the electronics 406 when a blood glucose monitor 414 is within range for wireless communication. Of course those of skill in the art will readily appreciate that the invention is not limited to any particular wireless technology, and could include, among other technologies, any custom or proprietary magnetic, RF, or infrared wireless communication protocol. The electronics 406 then establish a communication link with the blood glucose monitor 414, which allows data to be transferred to the output device 402 by wireless means. In one embodiment the blood glucose monitor 414 is adapted to provide an alert to the user which indicates that a connection with the output device 402 is available. By pressing a button on the blood glucose monitor 414, or otherwise indicating a desire to transfer data to the output device, a data transfer from the blood glucose device 414 to the output device 402 is caused.

The electronics 406 are further adapted to format printed pages based on the received SMBG data, generate graphical representations of data, such as pie charts, histograms, and the like, and to further format any information which is to be printed. The electronics 406 further drive the printer 404. Finally, the electronics 406 manage any other interfaces which may be provided with the system 400, such as a link to a PC or network for data storage or retrieval, configuration, software upgrades, and the like.

In one embodiment of the invention, the electronics 406 are further provided with logic to analyze the data received from a blood glucose monitor 414 and to apply therapy rules to the data. In this manner, the device is capable of providing therapy recommendations based on an analysis of the SMBG data, along with other printed matter such as graphical displays of the data, in order to provide therapy guidance to the patient and the healthcare provider.

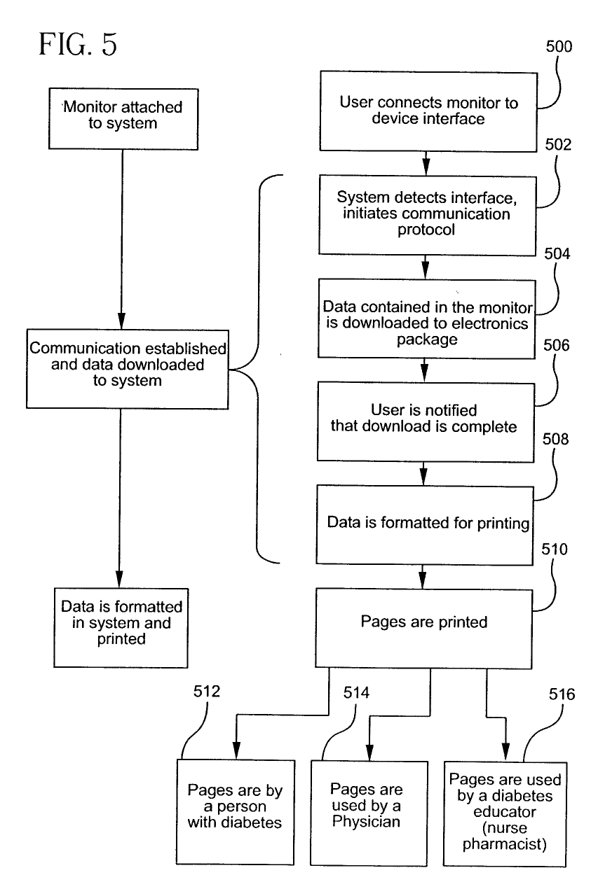

FIG. 5 is a flow chart illustrating the functionality of a system according to an embodiment of the present invention. The device 402 is preferably activated by connecting a monitor 414 to the device 402 at step 500. According to the preferred embodiment of the invention, this is the only action which must be taken by the user to produce an output. It will be recognized by those of skill in the art that a physical connection is not required, and data transfer may be accomplished by wireless means, such as for instance by the Bluetooth or 802.11b wireless protocols or through an infrared data port, for example. In a wireless embodiment, rather than physically connecting the monitor to a printing device, the user may only be required to place the monitor in proximity to a printing device. The monitor is adapted to recognize a suitable printer within its proximity and alert the user to the established communication between monitor and printer. In this case, the user simply indicates the desire for a data transfer to occur, as for example, by pressing an appropriate button provided on the monitor for this purpose.

Once the monitor 414 is connected, as described in connection with FIG. 2, the device 402 detects the monitor 414 and initiates a communications protocol with the monitor, at step 502, in order to download data from the monitor 414 to the device 402. At step 504, the data contained in the monitor 414 is downloaded to the electronics 406 of the device 402 to be analyzed and processed. The user is notified once the data transfer has been completed at step 506.

At step 508, the data which was received from the monitor 414 is analyzed and processed. The data is formatted for printing. Preferably, the formatting includes generating graphical displays of the data, such as predefined histograms, line charts, and any other graphical displays which might be helpful to the healthcare professional and the patient. In addition, during data processing, a set of therapy rules can be applied to the data, resulting in the output of a therapy recommendation. As an example, the data downloaded from the monitor 414 could include information indicating that the patient is receiving insulin twice per day. The data could further reveal that the patient experiences hypergycemia in the early afternoons (typically after lunch). Based on these factors, the printing device could apply therapy rules and generate a recommendation to start the patient on a three dose per day insulin program. In this manner a system according to the present invention can provide therapy guidance to the healthcare professional for the benefit of the patient. Of course the therapy rules applied by the system are limited only by the sophistication and processing power of the hardware, along with the level of detail of the software utilized by the system. A wide range of potential therapy rules are contemplated to be within the scope of the invention.

At step 510, the printer outputs printed pages containing the formatted data, such as graphical displays, logbook data, and therapy recommendations, as described above. Finally, at steps 512, 514, and 516, the printout can be used by a patient, a health care professional, or an educator. It should be understood that a paper printout is described in connection with the preferred embodiment of the invention, but the invention is not limited to a paper printout. Accordingly, an output to a CRT or LCD display, or the like, are contemplated to be within the scope of the invention.

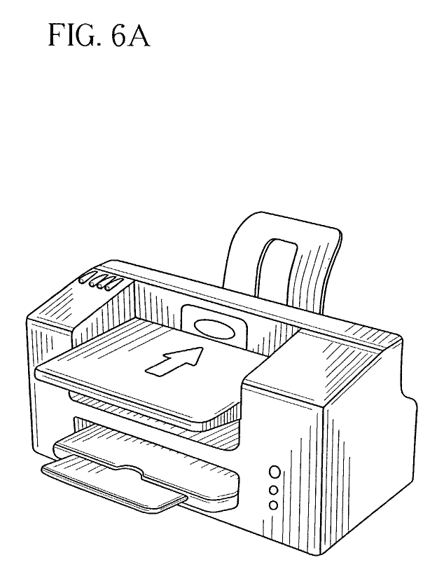

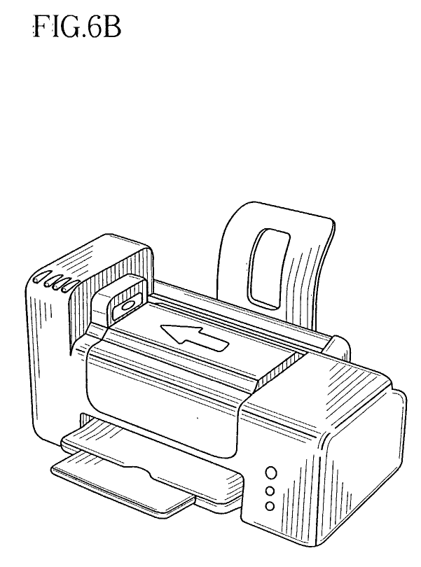

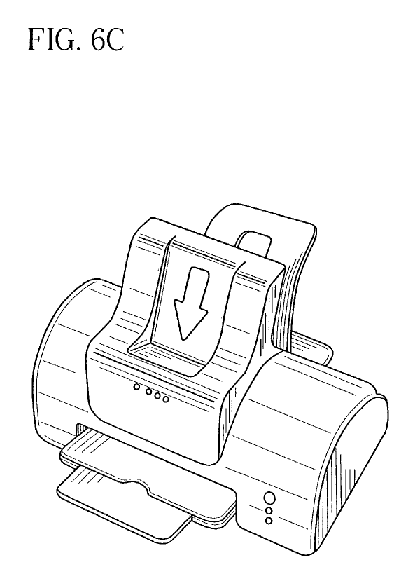

FIGS. 6A-6C illustrate alternate embodiments of a printing device. FIG. 6A illustrates an inkjet style printer with a front oriented connector for the body fluid chemistry analyzer. FIG. 6B illustrates another embodiment of the inkjet style printer with a side oriented connector for the body fluid chemistry analyzer. FIG. 6C illustrates an embodiment of the inkjet style printer with a top oriented connector for the body fluid chemistry analyzer.

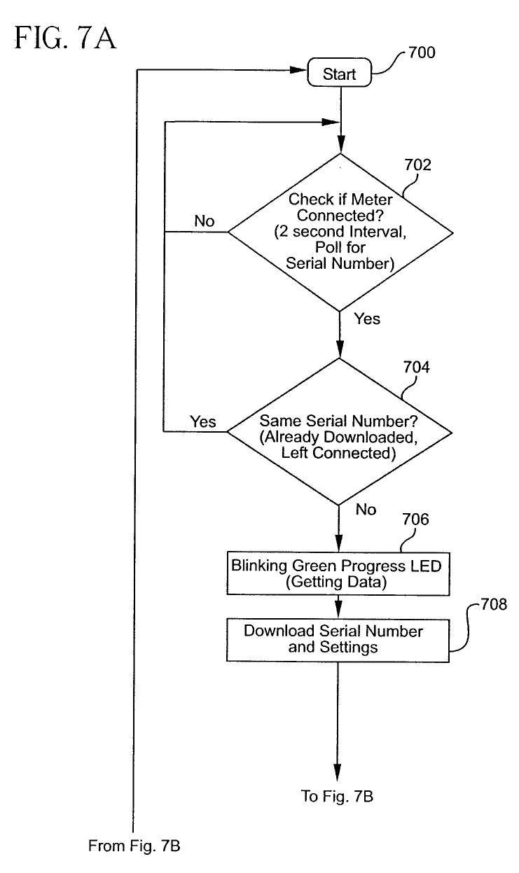

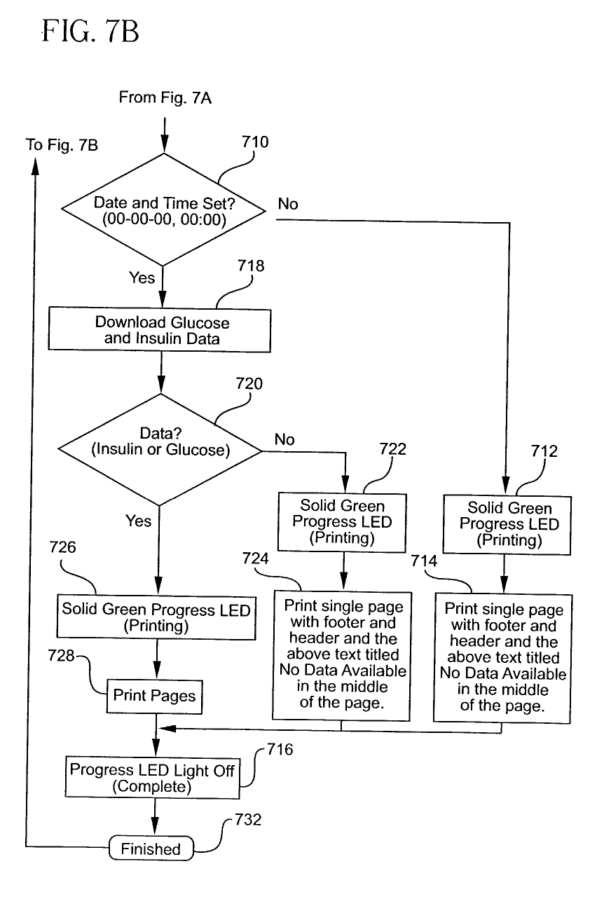

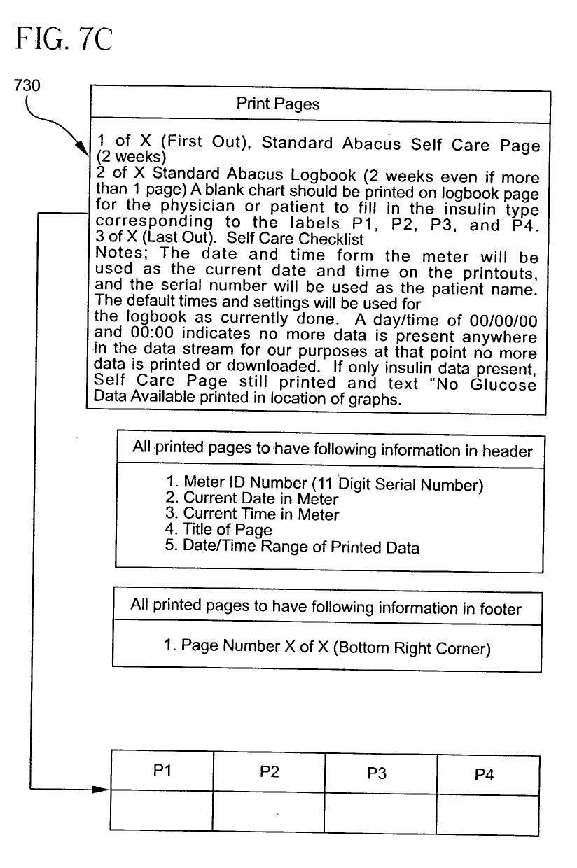

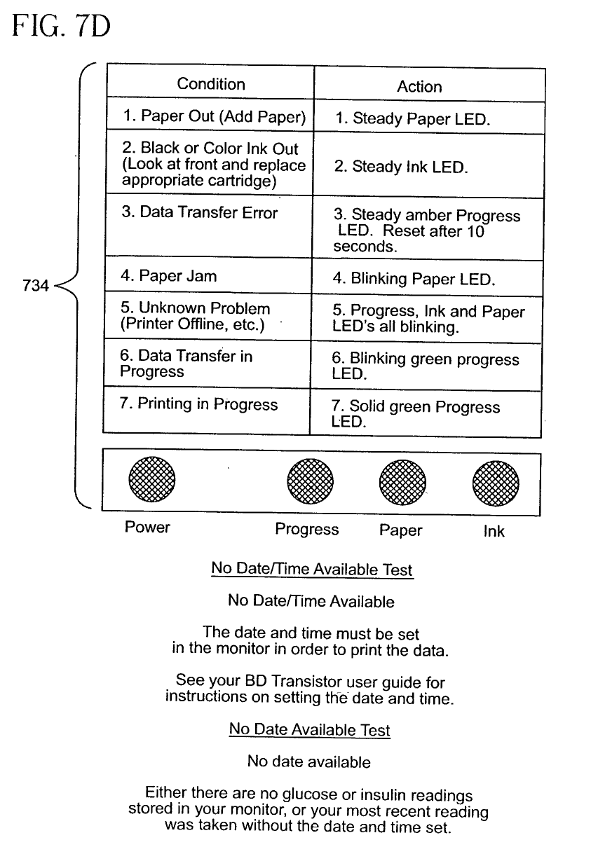

FIG. 7 is a more detailed flowchart illustrating the program flow of the processor controlling a printing device according to an embodiment of the invention. Program flow starts at step 700. At 702, the printer determines whether a meter is connected. If a meter is connected, the program determines the serial number of the meter, and whether it is a new serial number at step 704. If it is a new serial number, program flow continues to step 706. While data is being retrieved from the meter, an LED indicator is made to blink (step 706). The meter's serial number and settings are downloaded at step 708.

At step 710, the program determines whether the meter's date and time have been set. If the time and date have not been set, program flow continues to 712, where an LED is solidly lit while a page is printed at step 714. The page indicates that the time and date of the meter have not been set. Program flow then continues to step 716 to be described below. If during step 710, the program determines that the data and time of the meter have been set, program flow continues to step 718, where glucose and insulin data are downloaded. Next, at step 720, the program determines whether any glucose or insulin data was downloaded. If no data was downloaded during step 718, the program flow continues to step 722, in which an LED is solidly lit, while a page is printed at step 724. The printed page indicates that no data was available.

If during step 720, it is determined that glucose and insulin data was downloaded, program flow continues to step 726, in which an LED is solidly lit, while printed pages are produced at step 728. The format and content of printed pages is described generally at 730. Status and error conditions are also preferably indicated by controlling a set of LED's, as indicated generally at 734. At step 716, when all pages to be printed have completed printing, the progress LED is turned off. Program flow concludes at step 732, at which point program flow preferably loops back to the start, at step 700.

While the invention herein disclosed has been described by means of specific embodiments and applications thereof, numerous modifications and variations can be made thereto by those skilled in the art without departing from the scope of the invention.

What is claimed is:

1. A printing device for use with a body fluid chemistry analyzer, comprising:
a communication port adapted for communication with a body fluid chemistry analyzer; and
a printing device adapted to be controlled by a processor, wherein said processor is adapted to, in response to a single action by a user, retrieve body fluid chemistry data from said body fluid chemistry analyzer, format said data, and print said formatted data.

2. The printing device of claim 1, wherein said printing device has a set of drivers for interfacing with said body fluid chemistry analyzer pre-installed prior to delivery to an intended user.

3. The printing device of claim 1, wherein said single action comprises connecting said body fluid chemistry analyzer to said communication port.

4. The printing device of claim 1, wherein said body fluid chemistry analyzer comprises a blood glucose monitor.

5. The printing device of claim 1, wherein said communication port is a wireless communication port, said printing device further comprising a monitoring circuit adapted to poll for an available wireless communication device, to establish a communication link with said available wireless communication device, and to provide an alert to said user indicating the presence of said wireless communication device, and wherein said single action comprises responding to said alert.

6. The printing device of claim 1, wherein said wireless communication port comprises an infrared port.

7. The printing device of claim 1, wherein said wireless communication port comprises an IEEE 802.11 port.

8. The printing device of claim 1, wherein said wireless communication port comprises a Bluetooth port.

9. The printing device of claim 1, wherein said processor is further adapted to generate charts from said data, and to print said charts in response to said single action.

10. A method of retrieving and printing stored blood chemistry data comprising the steps of:
in response to a single action by a user, retrieving body fluid chemistry analyzer data from a body fluid chemistry analyzer through a communication port of a printing device,
formatting said data, and
printing said formatted data.

11. The method of claim 10, further comprising the step of pre-installing a set of drivers adapted to interface said printing device and said body fluid chemistry analyzer prior to delivery of said printing device to a user premises.

12. The method of claim 10, wherein said single action comprises connecting said body fluid chemistry analyzer to said communication port.

13. The method of claim 10, wherein said body fluid chemistry analyzer comprises a blood glucose monitor.

14. The method of claim 10, wherein said communication port comprises a wireless communication port, and said printing device further comprises a monitoring circuit adapted to poll for an available wireless communication device, to establish a communication link with said available wireless communication device, and to provide an alert to said user indicating the presence of said wireless communication device, and wherein said single action comprises responding to said alert.

15. The method of claim 10, wherein said wireless communication port comprises an infrared port.

16. The method of claim 10, wherein said wireless communication port comprises an IEEE 802.11 port.

17. The method of claim 10, wherein said wireless communication port comprises a Bluetooth port.

18. The method of claim 10, further comprising the steps of generating charts from said data, and printing said charts in response to said single action.

19. A computer readable medium of instructions adapted to control a printing device to retrieve and print body fluid chemistry data from a body fluid chemistry analyzer, comprising:
a first set of instructions adapted to control said printing device to receive a single action from a user; and
a second set of instructions adapted to control said printing device in response to said single action to retrieve body fluid chemistry analyzer data from a body fluid chemistry analyzer through a communication port of said printing device, to format said data, and to print said formatted data.

20. The computer readable medium of instructions of claim 19, wherein said second set of instructions comprises a set of drivers that are pre-installed prior to delivery of said printing device to an intended user.

21. The computer readable medium of instructions of claim 19, wherein said single action comprises connecting said body fluid chemistry analyzer to said communication port.

22. The computer readable medium of instructions of claim 19, wherein said bodily fluid chemistry analyzer comprises a blood glucose monitor.

23. The computer readable medium of instructions of claim 19, wherein said communication port comprises a wireless communication port, said printing device further comprising a monitoring circuit adapted to poll for an available wireless communication device, to establish a communication link with said available wireless communication device, and to provide an alert to said user indicating the presence of said wireless communication device, and wherein said single action comprises responding to said alert.

24. The computer readable medium of instructions of claim 19, wherein said wireless communication port comprises an infrared port.

25. The computer readable medium of instructions of claim 19, wherein said wireless communication port comprises an IEEE 802.11 port.

26. The computer readable medium of instructions of claim 19, wherein said wireless communication port comprises a Bluetooth port.

27. The computer readable medium of instructions of claim 19, wherein said second set of instructions is further adapted to generate charts from said data, and to print said charts in response to said single action.

28. A method of providing therapy guidance comprising the step of: providing a printer adapted for communication with a body fluid chemistry analyzer, wherein said printer is pre-configured such that upon establishing a communication link with said body fluid chemistry analyzer, said printer downloads data from said body fluid chemistry analyzer, analyzes said data, and prints a report based on said analysis of said data.

29. The method of claim 28 wherein said analysis comprises applying therapy rules to said data and generating recommendations based on said therapy rules and said data.

30. The method of claim 29 wherein said report comprises said recommendations.

31. The method of claim 28, wherein said printer needs only to be powered-on to be ready to use.

32. A printer adapted for communication with a body fluid chemistry analyzer, wherein said printer is pre-configured such that upon establishing a communication link with said body fluid chemistry analyzer, said printer downloads data from said body fluid chemistry analyzer, analyzes said data, and prints a report based on said analysis of said data.

33. The printer of claim 32 wherein said analysis comprises applying therapy rules to said data and generating recommendations based on said therapy rules and said data.

34. The printer of claim 33 wherein said report comprises said recommendations.

35. The printer of claim 32, wherein said printer needs only to be powered-on to be ready to use.
